(19) United States
(12) Patent Application Publication
(10) Pub. No.: US 2026/0207951 A1
(43) Pub. Date: Jul. 23, 2026

(54) APPARATUS, SYSTEMS, AND METHODS FOR PACING IN THE LEFT BUNDLE BRANCH BLOCK

(71) Applicant: Ablation Innovations, LLC, Brentwood, TN (US)

(72) Inventors: Daniel Walter Kaiser, Nashville, TN (US); Margaret Rox, Brentwood, TN (US)

(21) Appl. No.: 19/452,746

(22) Filed: Jan. 19, 2026

Related U.S. Application Data
(63) Continuation-in-part of application No. PCT/US2024/038889, filed on Jul. 19, 2024.
(60) Provisional application No. 63/527,785, filed on Jul. 19, 2023.

(51) Int. Cl. A61N 1/375; A61N 1/05

(52) U.S. Cl. CPC A61N 1/3756; A61N 1/0563; A61N 1/0565; A61N 1/0573; A61N 2001/058

(57) ABSTRACT

Leadless ventricular pacemaker devices are provided along with systems and methods for using them. In one example, the device includes an elongated member configured to be advanced through a tubular member into a patient's heart; a conformational change mechanism configured to reconfigure the elongated member from an elongated shape to a folded state upon exiting the tubular member; and a plurality of electrodes on the elongated member and configured to deliver unipolar or bipolar pacing to tissue within the patient's heart, wherein the device is configured to be implanted into a septum of a right ventricle within or adjacent to a native conduction system of the patient's heart.

### RELATED APPLICATION DATA

The present application is a continuation of co-pending International Application No. PCT/US2024/038889, filed Jul. 19, 2024, which claims benefit of U.S. provisional application Ser. No. 63/527,785, filed Jul. 19, 2023, the entire disclosures of which are expressly incorporated by reference herein.

### TECHNICAL FIELD

The present application relates to the field of cardiac resynchronization therapy (CRT) and to apparatus, systems, and methods for improving left bundle branch pacing (LBBB). More specifically, the present application relates to devices designed to facilitate accurate and consistent placement of a pacing lead within the left bundle branch, or leadless pacemaker devices aimed at improving the effectiveness of LBBB pacing and potentially improving clinical outcomes for patients with heart failure and other cardiac conditions.

### BACKGROUND

Left bundle branch pacing (LBBP) is a specialized form of cardiac resynchronization therapy that has gained increasing attention in recent years due to its potential advantages over traditional biventricular pacing (BiVP). While BiVP has been shown to improve outcomes in patients with heart failure, it is limited by its dependence on the location of the coronary sinus and its inability to directly activate the left bundle branch. LBBP, on the other hand, can provide more direct physiological activation of the left ventricle, potentially leading to improved outcomes and fewer complications. LBBB pacing involves the placement of a pacing lead in a specific location within the left bundle branch of the heart, which can be technically challenging.

The success of LBBB pacing is highly dependent on the accurate placement of the pacing lead within the left bundle branch. Currently, the placement of the lead relies heavily on the skill and experience of the operator, which can lead to inconsistencies in lead placement and suboptimal clinical outcomes. The use of LBBP has been limited by technical challenges related to the placement of the pacing lead within the left bundle branch. Traditional methods of lead placement, such as the use of fixed-curve catheters or steerable sheaths, have been associated with inconsistent and inaccurate lead placement, leading to suboptimal outcomes and complications.

Therefore, there remains a need for devices that can improve the accuracy and/or consistency of lead placement for LBBP, e.g., while providing optimal support and stability during the procedure, or a leadless pacemaker that can be inserted into the left bundle branch. The present application addresses this need by providing apparatus, systems, and methods that include an adjustable delivery sheath, selective stiffening, anchoring in the septal wall, steerable needle-like leads, and a permanently implantable leadless pacemaker for the LBBB, all to provide greater accuracy and consistency in LBBP. There is a need for devices that can aid in the placement of pacing leads or provide leadless pacing for LBBB pacing, providing a more consistent and reliable approach to this specialized form of cardiac resynchronization therapy. The systems and methods herein are designed to facilitate accurate and consistent placement of the pacing lead within the left bundle branch, making LBBB pacing more accessible and effective for patients with heart failure and other cardiac conditions. The following description provides a detailed overview of various examples of apparatus, systems, and methods.

### SUMMARY

The present application is directed to apparatus, systems, and methods for pacing the left bundle branch block (LBBB), directing leads or electrodes to the left bundle branch, performing cardiac resynchronization therapy (CRT), and/or delivering defibrillation to the heart via an implanted device. The systems and methods are designed to improve the accuracy and consistency of lead placement within the left bundle branch, and/or to provide leadless pacing in the left bundle branch, allowing for more effective and reliable LBBB pacing.

More particularly, the systems and methods herein may include a device that is implanted from within the left ventricle into the septal wall to access the left bundle branch block, paces the LBB, and provides CRT, and/or a device designed to facilitate more accurate placement of leads within the LBB for traditional pacing with leads. The device may also include systems and methods for monitoring the heart's natural rhythm, and for delivering electric shocks to restore regular heart rhythm.

The application includes a leadless pacemaker system comprising a steerable delivery sheath designed to facilitate precise placement of the pacemaker within the cardiac conduction system. The steerable delivery sheath may include a steerable mechanism and/or a curvature design. The delivery sheath is equipped with a steerable mechanism, allowing for fine-tuned manipulation and angulation to navigate the vasculature and heart chambers. The mechanism may include pull wires, steering knobs, or other actuating elements that provide the operator with precise control over the sheath's tip orientation. The sheath is designed with a pre-formed or adjustable curvature that enables it to align with anatomical pathways. The curvature is specifically configured to facilitate entry through the inferior vena cava (IVC) into the right atrium and subsequently into the right ventricle. The distal end of the sheath can be angled medially and slightly posteriorly to target the interventricular septum.

In one example, an adjustable sheath is provided that can be reconfigured in the left ventricle to access different regions of the septal wall for better placement of pacing leads or electrodes. The adjustable sheath may include tendons that can be tensioned to achieve directed bending of the sheath. The tendons may be connected to a control unit that allows the operator to adjust the tension in real time, providing greater control and precision during the procedure. Alternatively, other forms of actuation, such as shape memory alloys or hydraulic systems, may be used to achieve directed bending of the sheath. The sheath may also include a mechanism for stiffening, such as granular jamming, that can be activated to provide additional support and stability during the placement of the pacing lead. The granular jamming may be controlled by a valve or other means that allows the operator to adjust the stiffness of the sheath as needed.

In some examples, the adjustable sheath me be equipped with anchoring mechanisms that can be deployed in the septal wall for stability. The anchoring mechanisms may be expandable, allowing them to grip the tissue and hold the sheath in place during the procedure. The anchoring mechanisms may also be screw-like in nature and be twisted into the septal wall. The anchoring mechanisms can be retracted while the sheath is being adjusted and inserted when ready to deploy the pacing leads or electrodes.

The distal end of the leadless pacemaker incorporates an anchoring system equipped with one or more screws or pacing electrodes designed to secure the device within the myocardium. The anchoring system features screw anchors, pacing electrodes, and/or pacing morphology analysis. The distal anchoring system includes one or more extendable screws that can penetrate the myocardial tissue, providing stable fixation. The screws are designed to be deployed by rotational or linear motion controlled through the delivery sheath. Multiple pacing electrodes are integrated into the distal end of the pacemaker. These electrodes can be individually or collectively activated to deliver pacing stimuli. Each electrode is capable of delivering pacing signals, enabling the analysis of paced morphologies to determine optimal placement. By selectively pacing from different electrodes and analyzing the resulting paced electrogram morphologies, the operator can adjust the position of the delivery sheath. This adjustment ensures that the pacemaker is driven into the conduction system, achieving optimal pacing characteristics.

The delivery system includes various bends and curvatures to aid in the precise engagement of the conduction system. This may include any single bend or combination of bends. A primary bend near the distal tip of the delivery sheath is designed to navigate the sheath through the tricuspid valve into the right ventricle. This bend aligns the sheath with the interventricular septum. A secondary, more pronounced bend can be incorporated to adjust the angle of approach towards the septum, ensuring the distal anchoring system is optimally oriented for engagement with the conduction system. The delivery sheath allows for fine-tuning adjustments of its distal end to provide the necessary torque and directional control. These adjustments ensure that the pacing electrodes or screws can be accurately positioned within the conduction system. The delivery sheath can be provided in multiple configurations, each with distinct curvature profiles to accommodate different anatomical variations and procedural requirements.

This leadless pacemaker system with a steerable delivery sheath and an adjustable distal anchoring system offers enhanced precision in engaging the cardiac conduction system. The ability to analyze paced morphologies and adjust the sheath accordingly ensures optimal placement and pacing performance, significantly improving patient outcomes

Other aspects and features of the present invention will become apparent from consideration of the following description taken in conjunction with the accompanying drawings.

### BRIEF DESCRIPTION OF THE DRAWINGS

The invention is best understood from the following detailed description when read in conjunction with the accompanying drawings. It will be appreciated that the exemplary apparatus shown in the drawings are not necessarily drawn to scale, with emphasis instead being placed on illustrating the various aspects and features of the illustrated examples.

FIG. 1 shows an example of a lead delivery system to place pacing leads in the left bundle branch block.

FIGS. 2A-2D show an example of a delivery sheath or catheter that is capable of stiffening.

FIGS. 3A and 3B show an example of a leadless pacemaker system for the left bundle branch.

FIG. 4 demonstrates various components of this leadless pacing system once it has been implanted in the septal wall of the patient.

FIG. 5 shows a different view of the heart and how each of the components embed in the septal wall of the patient.

FIGS. 6A and 6B demonstrate placement of a catheter through the septal wall for HIS bundle or left bundle branch block placement.

FIG. 7 shows a fluoroscopic image of a right ventricle and HIS bundle.

FIG. 8 demonstrates an example of a leadless pacing system placed in the septal wall.

FIGS. 9A-9C show an example of the procedure for placing the pacing system in the septal wall.

FIGS. 10A-10C show another example of a pacing system being placed into a patient's heart.

FIGS. 11A and 11B show a different example in which pacing components fold into place before embedding in the septal wall.

FIG. 12 shows an example with different pacing components for the leadless pacing system.

### DETAILED DESCRIPTION

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

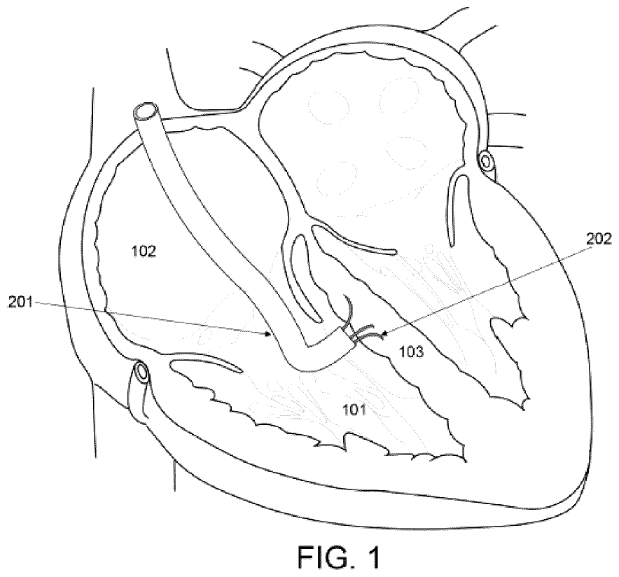

Turning to the drawings, FIG. 1 shows an example of a pacing lead delivery system including an adjustable outer sheath 201 and steerable needle-like leads 202 that are implanted in the septal wall 103. The sheath is delivered through the right atrium 102 and into the right ventricle 101 to reach the septal wall, in which the left bundle branch block is housed. The leads are intended to pace in the left bundle branch block of nerves for bi-ventricular pacing, which has better outcomes than single ventricle pacing. The distal end of 201 may include one or more features, e.g., a screw tip or other anchor (not shown), for securing the distal end of 201 relative to the heart, e.g., into the septum 103 between the right atrium 101 and the left atrium.

The lead placement device 202 is intended to pierce the septal wall to access the left bundle branch. The device 202 may include two or more needles that can be steered through one or more mechanisms such as a fixed precurvature imbued to an elastic material (such as nitinol or spring steel), two or more concentrically housed needles that are precurved to make a plurality of shapes to access the branch, flexible needles with a beveled tip that can interact with the septal tissue to curve and reach the branch, tendon-actuated needles with cuts to facilitate an alternative bending plane, and/or other features or methods to achieve steering not mentioned herein but understood by one of ordinary skill in the art. The methods described above may be facilitated through manual activation from the user, or through one or more motors, with or without a controller and user interface to direct the needle tips. In some examples, the needles are imbedded or may work in conjunction with sensing devices to direct the placement of the needle tip, such as but not limited to electromagnetic sensing, optical sensing, fiber-Bragg cables, acoustic or ultrasound sensing, or other forms of sensing not mentioned herein.

In another example of the lead placement device 202, one or more inner sheaths (not shown) are deployed from the outer sheath 201 to direct the lead placement device toward a desired location to pierce the septal wall 103 from within the right ventricle 101. The inner sheath(s) may be directed or steered using any of the methods described for actuating the outer sheath 201 or the lead placement needles.

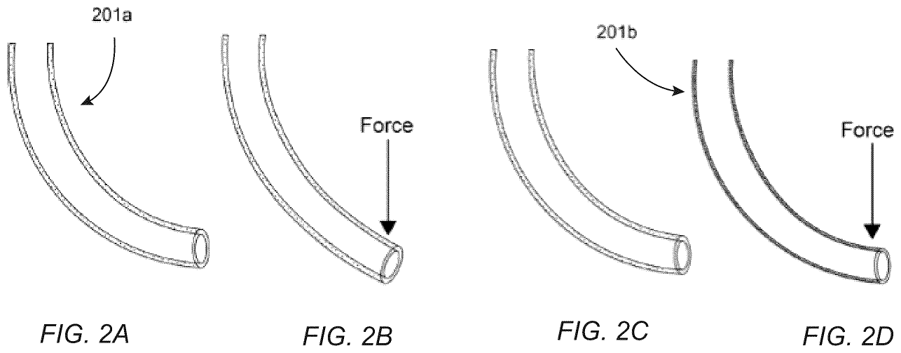

FIGS. 2A-2D show an example of a way the outer sheath 201 can be adjusted. The outer sheath, or catheter, 201 may include one or more features to make it adjustable, or it may be a fixed curvature to access the septum 103. In one example, there are tendons embedded in the sheath which can be tensioned to adjust curvature. The sheath may have cuts that facilitate bending in a certain direction or it may be capable of bending in any direction. In another example, there may be two or more concentrically housed sheaths of different curvatures and/or having cutouts with or without tendons to facilitate bending in a certain direction. Some examples may include a method of stiffening or selective stiffening, such as demonstrated in FIGS. 2A-2C with granular jamming, in which the sheath houses small granules between its inner wall and its outer wall which are loose while the sheath is being fixed in place (201a), and when desired, a vacuum removes the air between the granules to create a more rigid structure, which can withstand higher force (201b), as shown in FIG. 2D.

In some examples, the sheath is stiffened through methods such as using a low melting point alloy that can be rapidly heated to liquid form and cooled to solid form, or using another thermo-responsive material, such as hydrogel. In other examples, concentrically housed sheaths with redundancy in their actuation could use redundancy resolution to assemble in configurations with lower levels of compliance. The outer sheath 201 may be operated manually by the user or be controlled by one or more motors with or without a controller and/or user interface.

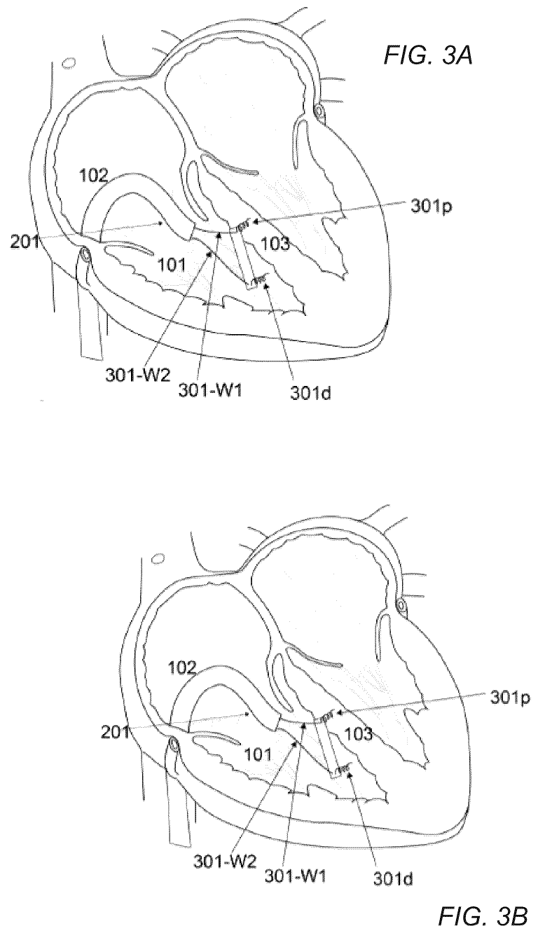

Turning to FIGS. 3A and 3B, an example of a leadless pacing system for the left bundle branch is demonstrated. The outer sheath 201 may include some, all, or none of the features described previously for the lead delivery system. In one example, the leadless pacemaker device 301 deploys from the outer sheath 201 into a superior location in the septal wall 103 and anchors with a mechanism, e.g., a screw tip or other anchor, 301p. The proximal end of device 301 (i.e., the end that exits the outer sheath first and embeds in the superior location of the septal wall) is connected via a wire, sheath, or needle 301-W1 to aid in placement of the device and/or to deploy or screw the anchor. The distal end is then released from the sheath to anchor into a posterior location of the septal wall 103 via the anchoring mechanism 301d. Another wire or sheath 301-W2 may assist in the placement of the distal end or in the deployment of distal anchor 301d. In some examples, there is a hinge mechanism on the proximal end of device 301 that enables the device to swing down to lay flat against or parallel to the wall of the septum. In other examples, the device may configure to this flat or parallel location automatically, via a mechanism such as a tension spring or precurved nitinol or other elastic material. Some examples may enable the release of the placement wires 301-W1 and 301-W2 through applying current or heat to break the connection. Alternatively, the wires may unscrew or have a releasement mechanism that can be activated by the user.

In another example, there may be an additional component (not shown) that assists in the configuration of the device 301 to lay against the septal wall 103. This may include a variety of mechanism but is not limited to the descriptions listed herein. In one example, a gripper mechanism or other device capable of grabbing the device is attached and used to fix the device 301 into place. In other examples, motors assist in rotating the device into the correct configuration. There may be more than two placement wires, sheaths, or needles, (like 301-W1 and 301-W2) that the user is able to control to manipulate the positioning of the device.

The leadless pacing device 301 is equipped with a battery, pacing circuitry, and electrodes. Optionally, the device may be equipped with one or more sensors to measure physiological parameters such as but not limited to heart rate, blood pressure, blood oxygen level, and/or electrical signals. These sensors may include optical sensors, pressure sensors (piezoresistive, capacitive, inductive, impedance-based, or otherwise), acoustic sensors, or other sensors not listed herein.

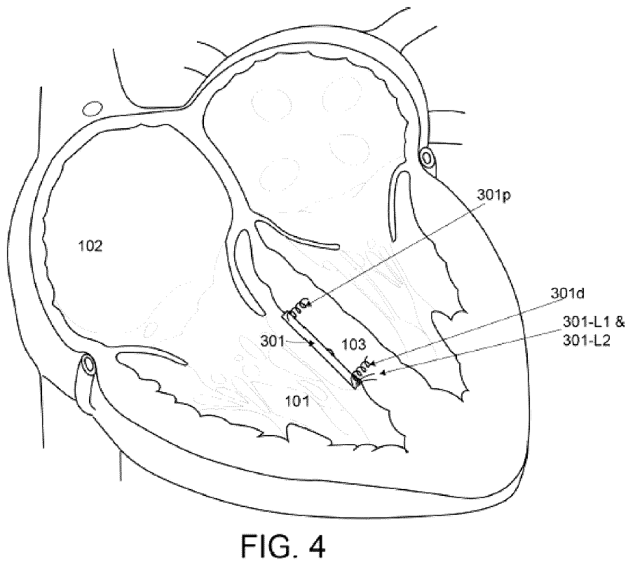

FIG. 4 is an example of the leadless pacemaker device when it is fully inserted into the patient heart. In one example, the pacemaker device 301 is parallel to the septum 103 when fully inserted. Anchors 301p and 301d are fully deployed and hold the device securely within the heart. The device 301 may be imbued with a substance to promote endothelialization. In some examples, electrodes 301-L1 and 301-L2 may be placed on the distal end (posterior of the septal wall) on either side of the anchor to attempt to capture both ventricles and ensure placement with the left bundle branch block. These or other electrodes (not shown) may also be placed at other locations along the device. The electrodes may be shaped like needles to pierce the septal wall, may be curved or steerable, and/or may be another shape not listed herein.

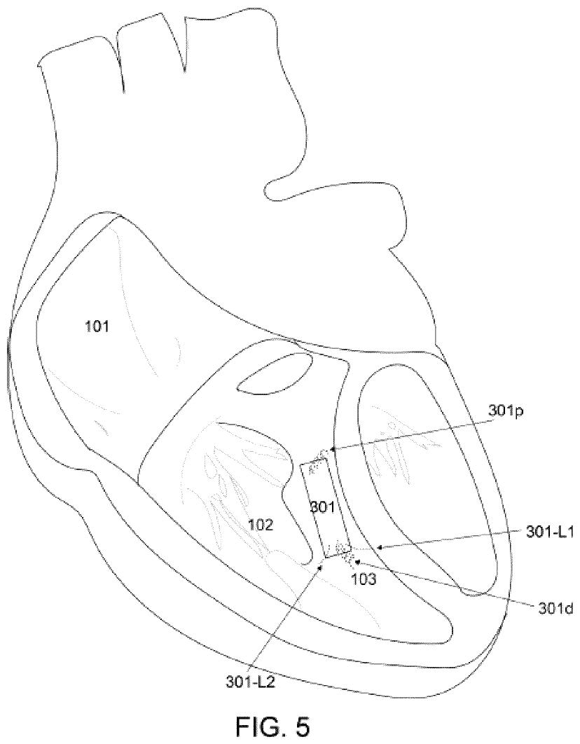

FIG. 5 demonstrates an example with a rotated view of the heart to see more of the septal wall. In some examples, the distal end of the device is capable of being rotated left and right (from the view of the figure). This will enable the user to test capture locations and place electrodes 301-L1 and 301-L2 in optimal locations, or other electrodes (not shown), before fully anchoring. Electrodes may be spaced to test optimal capture. In some examples the device includes leads (not shown) attached to the device 301 that are capable of shocking the heart and acting as a cardioverter defibrillator in emergency situations. Leads may be placed on or in any combination of the ventricles, in the septum, in the coronary sinus, or in another location within the heart.

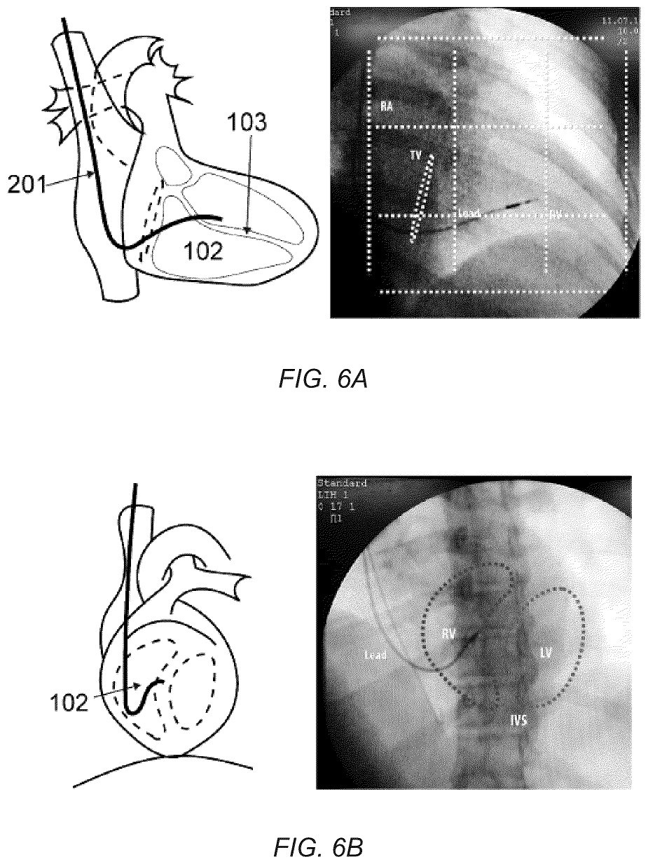

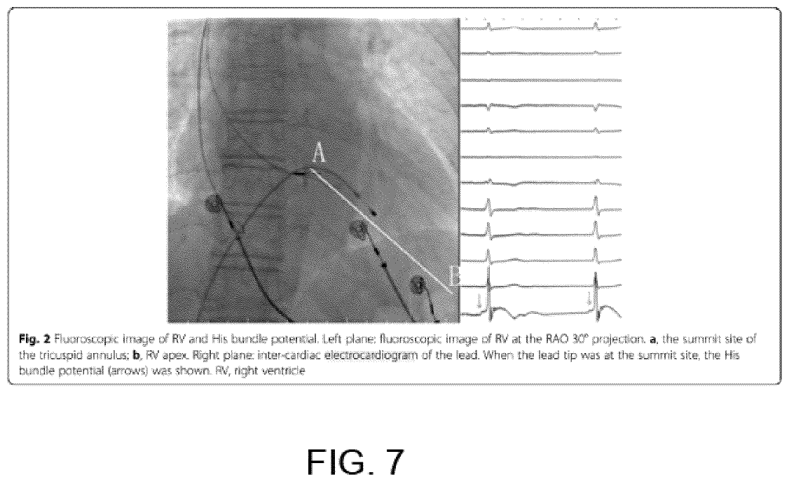

FIGS. 6A and 6B show two planar views of the heart with both line drawing and fluoroscopy in which a catheter 201 is deployed into the right ventricle (RV) to pierce the septal wall for placement of a leadless pacemaker. FIG. 7 demonstrates another fluoroscopic image of the RV with an inter-cardiac electrogram of the lead, demonstrating what certain examples of this procedure may entail.

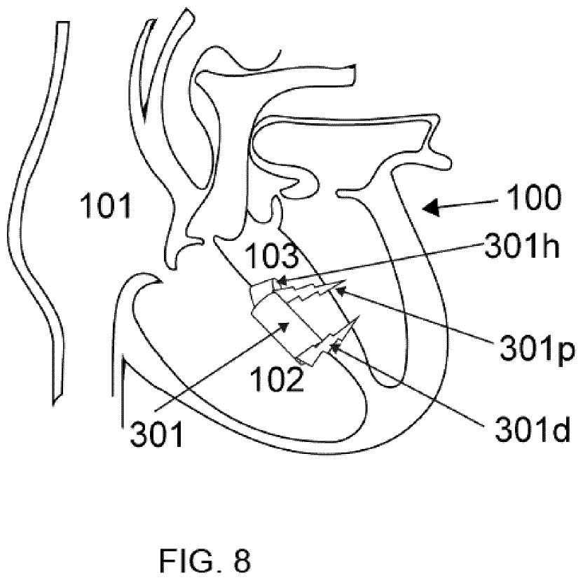

FIG. 8 shows an example of the leadless pacemaker 301 embedded in the septal wall 103 of the heart 100 after feeding the catheter into the RV 102. The pacemaker 301 features a hinge mechanism 301h to ensure flat placement against the wall and both a proximal (301p) and distal (301d) anchor and/or lead and/or electrode. Other examples may include more pacing leads, electrodes, or anchoring screws or tines along the system.

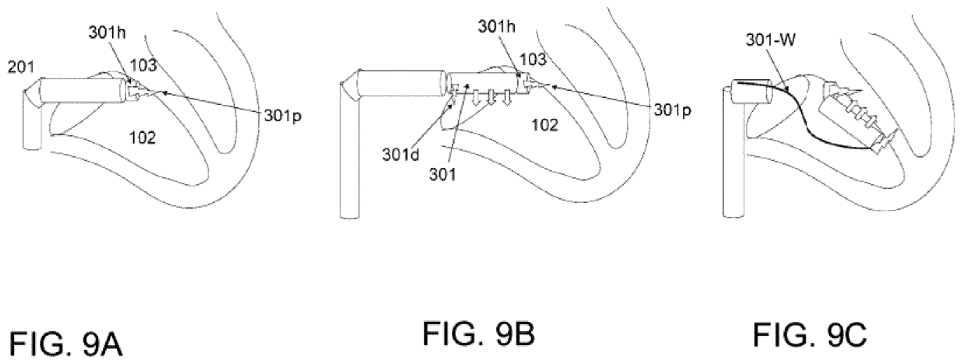

FIG. 9A demonstrates a distal rotational tool initially used to rotate the device and screw distal screw into the high septum 201. After anchoring, pulling this back enables the device to be rotated with hinge 301h into the correct position to enable the device to fold longitudinally towards the distal interventricular septum. In FIG. 9B more of the sheath 201 is pulled back to expose the rest of the device 301. FIG. 9C shows how pushing deployment wire 301-W forces the device into the distal interventricular septum. In some examples, this wire is rotated to anchor the screw the distal end into the septum. This wire is then released to leave the device within the right ventricle.

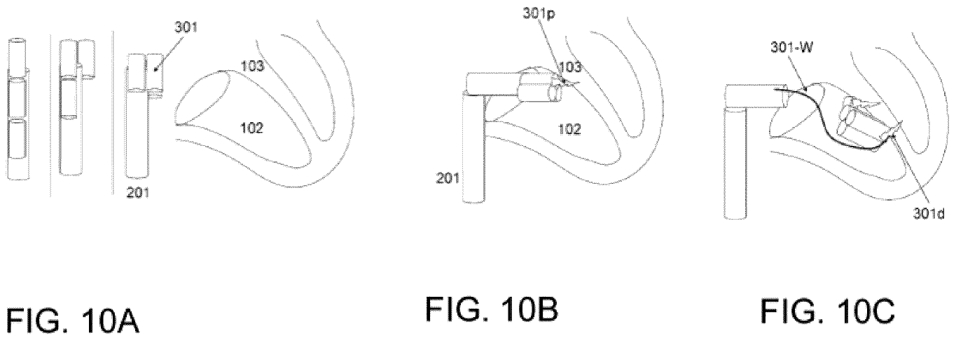

In some examples, the device rotates prior to anchoring. In this example, the device folds several times within the right atrium, SVC, IVC, or right ventricle. This folded device is then deployed into the right ventricle. FIG. 10A shows the device 301 rotating out of sheath 201 to create a structure that is then deployed into the RV 102 and rotated to lie against the septal wall 103 in FIG. 10C.

In one example, the leadless ventricular pacemaker device 301 may include an elongated member that is advanced through a catheter and housed in a delivery sheath (not shown). Upon exiting the sheath, a conformational change mechanism reconfigures the member from an elongated shape to a folded state, optimizing placement and anchoring within the right ventricle septum, e.g., as shown in FIG. 10A. The device includes a plurality of electrodes, which can be paced unipolar or bipolar between the various electrodes. These electrodes can be deployed sequentially into the septal tissue to ensure proper anchoring and contact, capturing the heart's conduction system for a narrow paced QRS complex.

In some examples, the device 301 includes an elongated member that is advanced through a catheter and/or delivery sheath (not shown) and deployed near or within the right atrium. Upon exiting the delivery sheath, the device undergoes a conformational change to facilitate anchoring and optimal positioning within the RV septum. As the elongated member exits the delivery sheath, the device 301 transitions from a compact to a reconfigured state. This transformation is achieved through a mechanical design that responds to the sheath release, ensuring correct alignment with the RV septum for optimal electrode placement. This mechanism minimizes the device's profile during delivery and ensures robust anchoring and electrode contact once deployed.

Optionally, the system may include at least three electrodes designed to penetrate and embed into the tissue; the system able to deliver electrical pulses to any of the electrodes in a variety of pacing configurations. Precise electrode placement may be useful for a narrow-paced morphology, essential for synchronizing the heart's electrical activity and optimizing cardiac output. The anchoring system ensures stable, long-term fixation, preventing migration and ensuring consistent pacing performance. Optionally, these electrodes may be covered with steroids or other drug therapy to facilitate function. In other examples, electrical current is delivered to the anchoring system.

Various methods can be utilized to help anchor the devices described herein to the tissue. For example, thermal anchoring can be achieved through various methods, such as any of the following. Resistive heating involves delivering electrical current to the anchoring portion, generating heat via electrical resistance to melt or ablate tissue for a secure bond. Radiofrequency (RF) ablation utilizes RF energy delivered through an electrode to cause localized heating and tissue modification for anchoring. Laser ablation directs laser energy at the anchoring portion for precise tissue ablation and adhesion. Ultrasound ablation employs focused ultrasound energy to generate heat at the anchoring site, promoting tissue welding or ablation.

In addition or alternatively, adhesive anchoring may be used, such as any of the following. For example, heat-activated adhesives may be pre-applied to the anchoring portion and activated by an external heat source like electrical current, RF, laser, or ultrasound to create a strong bond with the tissue. Light-activated adhesives are photosensitive and cured using specific wavelengths of light for secure attachment. Chemical adhesives are biocompatible coatings that form a bond upon tissue contact or after a chemical reaction.

In addition or alternatively, mechanical anchoring may be used, which relies on physical structures. Barbed or tined anchors feature projections that penetrate and grip the tissue for mechanical stability. Expanding anchors increase contact area with the tissue upon deployment, enhancing fixation. Screw-type anchors are threaded and screwed into the tissue for a secure and stable attachment.

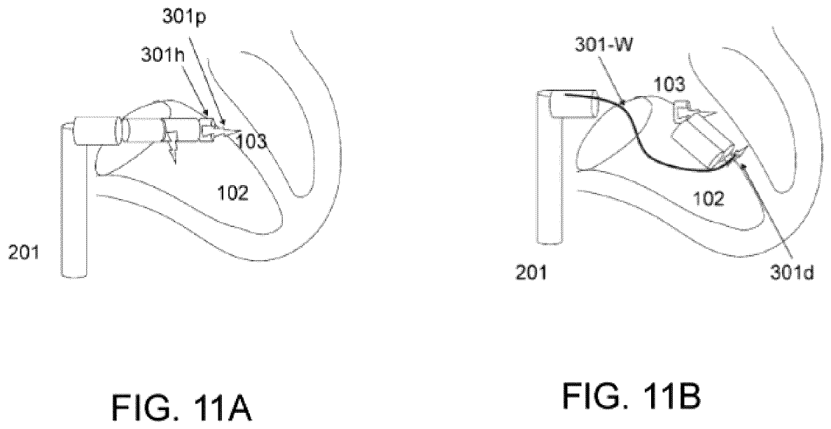

In some examples, the device rotates prior to anchoring. In this example, the device folds several times within the right atrium, SVC, IVC, or right ventricle. This folded device is then deployed into the right ventricle, as shown in FIGS. 11A and 11B.

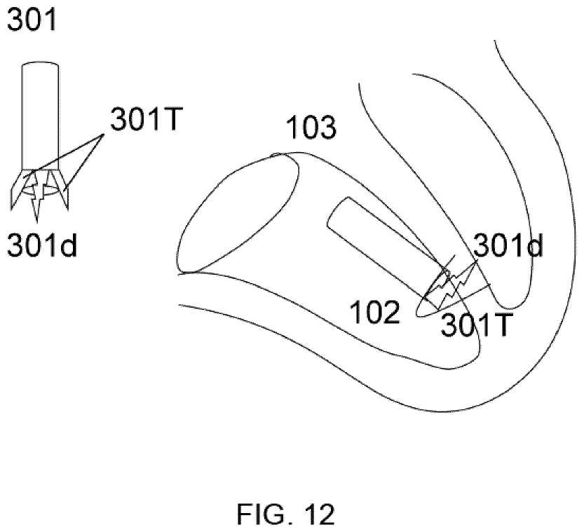

FIG. 12 demonstrates an example where the distal screw (and/or the proximal screw) when drilling into the interventricular septum may be flanked by tines (possible barbed) 301T. These tines help anchor the device and can be used to deliver pacing potentials. This increases the probability the heart's native conduction system is capture for a narrow paced QRS.

In another example, the device has steerable needles that can be deployed to place leads or electrodes for traditional or leadless pacing. The needles may be passively steerable or precurved, e.g., out of an elastic material like nitinol or spring steel, to deploy when they are released from the sheath or catheter. They may be steered to follow the curvature of the left bundle branch and/or curve through the tissue of the septal wall to reach the left bundle branch block. The needles may be rotated and inserted to adjust positioning, or have adjustable curvature in order to access a different part of the septal wall for optimal placement of the leads or electrodes.

Some examples may include a leadless pacemaker configured to lie flat against the septal wall. The body of the pacemaker may include a battery or multiple batteries to power the device. It may also include capabilities like Bluetooth or other communication protocols for monitoring heart signals and communicating with the device. The body may also include various sensors to measure pressure, oxygen, heartrate, or other physiological parameters for monitoring patient health.

In one example, one end of the pacemaker body (the proximal end) can be anchored into the top of the septal wall near the right atrium via a screw, hooks, tines, or other anchoring mechanisms. The anchoring mechanism may be screwed or otherwise fixated with an independent wire or sheath that is attached to the mechanism. The remainder of the pacemaker swings down via a hinge or segment of nitinol with precurvature to lay the device flat against the septal wall. The other end (the distal end) may also be anchored into the septal wall near the bottom of the right ventricle with a screw or other anchoring mechanism. The anchoring mechanism may be screwed or otherwise fixated with a separate independent wire or sheath. The independent wires or sheaths may be separated from the pacing device by a cutting mechanism or by applying electric current to terminate the connection.

In some examples, the pacemaker may include one or more pacing electrodes at the anchoring points or along the portion that lays against the septal wall. During placement, the electrodes may be tested for correct placement, and the lateral positioning of the distal end may be adjusted through a variety of methods by un-fixing the distal anchor. The independent wire or sheath on the distal end may be used to steer the device to a new angle, or the proximal end may be rotated in order to rotate the entire device into position. In other examples, there are additional positioning devices that deploy once the body of the pacemaker has been anchored into the septal wall. This may include steerable needle-like devices with embedded electrodes, or screw mechanisms that can be angled or swiveled into place to adjust pacing electrode positioning.

In other examples, the implantation process initial anchor is deployed in the high interventricular septum. This mechanism includes either a screw mechanism or tine-based system for initial fixation. This detailed description outlines the steps involved in the implantation, highlighting the use of fluoroscopy and intracardiac echocardiogram (ICE) for precise placement.

In this example and others, the deployment may act as follows. Advancing the Device into the Right Atrium: The leadless pacemaker, preloaded into a specialized delivery catheter, is introduced into the patient's venous system through a percutaneous approach. The catheter is carefully navigated through the vasculature, entering the right atrium via the inferior or superior vena cava. Throughout this process, fluoroscopic guidance ensures accurate navigation and positioning. Crossing the Tricuspid Valve: With the catheter securely positioned in the right atrium, the next step involves crossing the tricuspid valve. This is a critical phase, as the catheter must be maneuvered with precision to avoid damaging the valve or surrounding cardiac structures. Fluoroscopy provides real-time imaging, allowing the operator to visualize the catheter's movement and ensure safe passage into the right ventricle. Positioning in the High Interventricular Septum: Once in the right ventricle, the catheter is advanced towards the high interventricular septum, the target location for initial device implantation. The high interventricular septum is chosen due to its strategic location, offering optimal conditions for electrical stimulation of the ventricles. ICE imaging complements fluoroscopy by providing detailed views of the septal wall, aiding in precise catheter positioning. Anchoring Mechanism Screw or Tine-Based: The leadless pacemaker is equipped with a secure anchoring mechanism, essential for stable implantation.

In some examples, the initial deployment may include a variety of mechanisms to deploy the initial anchoring mechanism, including but not limited to: Helical Screw, in which the mechanism includes rotating to embed into septal tissue and a deployment tool is torque wire or inner rotation mechanism, Self-Tapping Screw, in which the mechanism includes cutting its own thread during insertion and the deployment tool is similar to helical screw, flexible tines (Medtronic Micra Style), in which the mechanism includes extending to grip tissue upon release and the deployment tool involves delivery catheter releases tines, Retractable Tines, in which the mechanism includes extending and retracting as needed and the deployment tool is controlled by delivery catheter, Barbed Hooks, in which the mechanism includes penetrating and gripping tissue, and the deployment tool includes pressing into the septal wall, Expandable Spikes, in which the mechanism includes expanding outward to embed and the deployment tool includes a mechanism to expand spikes, Balloon-Expandable Anchors, in which the mechanism includes a balloon inflating to deploy anchors and the deployment tool includes an integrated balloon, Screw with Tines, in which the mechanism includes combining screw rotation with tine grip and the deployment tool includes a torque wire and tine release, Expandable Screw, in which the mechanism includes expanding a base after insertion and the deployment tool includes a screw rotation and expansion mechanism, or another mechanism not mentioned here.

Some examples may include the use of rotation mechanisms. Once the anchor is deployed along the superior aspect of the interventricular septum, the device is then released to orient itself largely parallel with the interventricular septum. Various mechanisms are described to have the device orient itself along the ventricle, including but not limited to:
Spring-Loaded Rotation: In one example, the device includes a spring-loaded mechanism that stores mechanical energy during the initial deployment. Upon activation, the stored energy is released, causing the device to rotate towards the apex of the right ventricle. This rotation aligns the elongated body of the pacemaker parallel to the interventricular septum.
Nitinol Hinge: In one example, the device is equipped with a nitinol hinge that utilizes the shape-memory properties of nitinol. The hinge is pre-set to bend the device towards the septum when it reaches body temperature or upon application of a specific trigger. This allows the device to rotate into the correct orientation naturally.
Torque Wire Mechanism: In one example, the device incorporates a torque wire that extends from the delivery catheter. The clinician can apply rotational force through the torque wire, manually guiding the device to rotate into the desired position along the interventricular septum.

These and other examples may also include positioning and advancement mechanisms, including but not limited to:
Hinge and Slide Mechanism: In one example, after the initial rotation, the device features a hinge and slide mechanism that allows the body to pivot and slide along the septal wall. The hinge at the proximal end facilitates the initial rotation, and the sliding mechanism ensures the device aligns and lays parallel to the septum.
Guide Wire and Sleeve System: In one example, the device is advanced and positioned using a guide wire and sleeve system. The guide wire is threaded through the septum, and the pacemaker is advanced over the guide wire. Once the device reaches the target area, the sleeve is retracted, allowing the device to lay parallel to the septal wall and bring the distal anchoring system into place.
Expandable Segment: In one example, the pacemaker includes an expandable segment that extends and stabilizes the device against the septal wall. After the initial anchoring at the proximal end, the expandable segment deploys, pushing the device to lay flat along the septum. This segment can be activated mechanically or through an inflatable mechanism.
Controlled Deployment System: In one example, the device utilizes a controlled deployment system where the distal end is initially compacted. Upon reaching the correct position, a release mechanism activates, allowing the device to expand and lay along the septal wall. The deployment is guided by fluoroscopic and ICE imaging to ensure accurate positioning.
Magnetic Guidance: In one example, the device includes magnetic elements that interact with an external magnetic field to guide and position the pacemaker. The external magnetic system provides directional force, rotating and aligning the device parallel to the septal wall.

These and other examples may incorporate some of the following distal anchoring concepts. Simultaneous with positioning the leadless pacemaker along the interventricular septum, or using a separate step, the distal end of the leadless pacemaker is anchored along the distal aspect of the interventricular septum. In one example, the distal end of the leadless pacemaker is anchored using a single screw mechanism. This screw is designed to embed securely into the interventricular septum, providing both stability and electrical contact. The pacemaker is advanced into the desired position along the septum, and the screw is rotated via an internal drive mechanism within the device or through external control via the delivery catheter. The helical design of the screw allows it to penetrate the septal tissue with minimal resistance, ensuring a firm anchorage. This mechanism not only stabilizes the device but also aligns it properly for effective pacing.

In another example, the device employs a barb system for distal anchoring. This system consists of multiple barbs that extend from the distal end of the pacemaker. Upon deployment, the barbs are driven into the septal tissue, where their angled design prevents back-out and ensures secure fixation. The barbs can be extended either mechanically through a deployment actuator or by the natural expansion of a compressed state once the device is in position. This mechanism offers a reliable anchoring method that conforms to the septal tissue, providing both stabilization and pacing capabilities.

In one example, the distal anchoring mechanism uses a tine-based system similar to the Medtronic Micra. The distal end of the pacemaker is equipped with flexible tines that deploy upon reaching the target location. These tines penetrate several millimeters into the interventricular septum, gripping the tissue firmly. The deployment of the tines can be controlled via an external actuator in the delivery catheter, ensuring precise positioning. The tines' flexibility allows them to conform to the septal tissue, providing a stable anchor that can also facilitate effective ventricular pacing.

In yet another example, the device features a three-prong anchoring system. This system consists of three distinct tines, each equipped with a pacing electrode, which are advanced into the interventricular septum. The prongs are deployed sequentially or simultaneously, ensuring that they penetrate the tissue at different angles for maximum stability. The prongs not only anchor the device but also serve as pacing electrodes, enabling direct engagement with the heart's conduction system for effective pacing. The deployment of the prongs can be controlled via an internal mechanism or an external actuator, providing flexibility and precision during implantation.

In one example, the distal end of the pacemaker may utilize expandable spikes for anchoring. These spikes remain retracted during the initial positioning of the device. Once the pacemaker is correctly positioned along the septum, the spikes are expanded outward, embedding into the septal tissue. This expansion can be controlled mechanically or via an inflatable mechanism within the device. The expandable spikes offer a broad contact area, ensuring a secure and stable anchor that supports the pacemaker's pacing function.

In another example, the delivery system employs a hydraulic deployment system to push the distal anchors into the interventricular septum. This system uses a fluid-driven actuator within the delivery system that, when activated, extends the anchors into the septal tissue. The hydraulic pressure ensures controlled and uniform deployment of the anchors, which can be spikes, tines, or barbs. This mechanism provides a robust anchoring method that adapts to varying tissue densities and ensures precise positioning of the device.

In one example, the delivery system includes a hydraulic mechanism that incorporates a balloon for precise and controlled deployment of the distal anchors. The hydraulic mechanism may work by inflating a balloon within the delivery catheter or by engaging a fluid-driven actuator mechanism. After the distal end is flipped and aligned, the hydraulic system can also be used to advance screws or tines into the septum. The hydraulic pressure ensures that the screws are rotated into place or the tines are driven securely into the tissue. The ability to control the deployment force and depth enhances the reliability and effectiveness of the anchoring process.

In one example, the distal end of the leadless pacemaker is anchored using a combined screw and tine mechanism. This innovative approach enhances the stability of the pacemaker and provides multiple points for effective pacing. The Distal Screw Mechanism is the primary anchoring component at the distal end of the pacemaker which may include a helical screw designed to penetrate the septal tissue. The screw is advanced into the septum by rotating it via an internal drive mechanism within the device or through external control via the delivery catheter. The rotation embeds the screw securely into the septal wall. This screw provides a strong, central anchor point, ensuring that the pacemaker remains firmly in place.

This example and others may include flanking tines. Flanking the central screw are multiple tines, which serve both as additional anchoring points and potential pacing electrodes. As the screw is driven into the septum, the tines are simultaneously or sequentially deployed into the septal tissue. This deployment can be facilitated by the same rotational mechanism or a separate actuator within the delivery system. The tines penetrate several millimeters into the septal wall, providing additional stability to the device. They are designed to conform to the septal tissue, enhancing the overall fixation of the pacemaker.

The combination of the central screw and flanking tines ensures robust anchoring. The screw provides a central, deep anchor, while the tines distribute the anchoring force across a broader area, reducing the risk of device migration or dislodgment. In addition to their anchoring function, the tines can be used as pacing electrodes. Each tine is equipped with an electrode capable of delivering electrical stimulation to the septal tissue. This configuration allows for targeted pacing at multiple locations, optimizing the capture of the heart's conduction system and achieving a narrowly paced QRS complex.

For deployment, the pacemaker is advanced through the delivery system into the desired location along the interventricular septum. The deployment process begins with the rotation of the central screw, embedding it into the septal tissue. Once the screw is securely in place, the flanking tines are deployed. This can be achieved through the continued rotation of the screw mechanism or by activating a separate actuator that drives the tines into the septum. The deployment is guided by fluoroscopy and intracardiac echocardiogram (ICE) to ensure precise positioning and adequate penetration of both the screw and tines.

The dual anchoring system provides exceptional stability, reducing the likelihood of device migration or dislodgment. Multiple pacing electrodes enhance the device's ability to capture the heart's conduction system, ensuring effective and efficient pacing. The use of fluoroscopic and ICE guidance allows for real-time visualization, ensuring accurate deployment and positioning of the pacemaker.

The deployment mechanism can include depth control features to prevent over-penetration of the screw and tines, ensuring they reach the optimal depth for anchoring and pacing. The configuration of the tines can be adjusted based on the patient's anatomical and physiological requirements, providing flexibility and adaptability in various clinical scenarios. By incorporating a central screw flanked by tines, the leadless pacemaker achieves a secure and stable fixation in the interventricular septum, enhancing both its anchoring and pacing capabilities. This combined mechanism ensures that the device remains in place while delivering effective cardiac resynchronization therapy.

In one example, the pacing electrodes may penetrate approximately 2-4 millimeters into the septal tissue. This depth is sufficient to ensure stable fixation and effective electrical contact with the myocardial tissue, thereby enhancing the pacing performance. Deeper penetration of up to 5-7 millimeters may occur in some cases, but the design aims to prevent excessive penetration that could necessitate re-implantation.

Optionally, the devices herein may have an exposed screw that is screwed into the interventricular septum. For example, the entire device may be rotated to drive in this screw. Impedance and injury current can be analyzed to determine whether the device is adequately embedded or adhered to the tissue. A tug test may also be performed. After the superior or proximal anchor is adequately deplored, the rotating deliver agent can be withdrawn to free the distal end. With the distal attachment disengaged, turning this mechanism can be used to rotate the proximal aspects of the device. This rotating can be done to position the device in the correct orientation thus that the device will flip or rotate towards the interventricular septum or apex of the right ventricle.

A deployment wire can be used where pushing this wire swings the device into the septum. Significant force can be delivered by pushing this wire. With the device fixed superiorly, the whole device will rotate along this fulcrum into the more distal, lower aspect of the interventricular septum.

In one example, the elongated member undergoes a conformational change after leaving the deployment sheath in the right atrium. The device folds on itself several times to create several subunits relatively parallel to each other. The folded device can then be advanced and anchored into the high septum of the right ventricle. This anchor may be an electrode or have an adjoining electrode. Using a hinge or nitinol connection, the folded device can rotate towards the apex of the right ventricle. In some examples, one or more components of the device can extend towards the apex of the right ventricle. The device can deploy several electrodes and/or anchors along the device from the right ventricular septum all the way to the apex of the right ventricle. Therefore there are several electrodes with the potential of capturing the native conduction system of the heart. In some examples, the device can extend in order to space the electrodes. This is particularly important if defibrillation is delivered from the device in order to get a defibrillation as far apart as possible to capture as much of the myocardium as possible. In some examples, the device has a curve that matches the curve of the right ventricular septum.

In some examples, the anchors are positioned such that they secure the elongated member at a minimum of two locations, with the anchoring mechanisms spaced apart between approximately ten and forty millimeters (10-40 mm). This specific spacing ensures that the pacemaker maintains stable contact with the septal tissue, preventing dislodgement while delivering consistent pacing signals. The multiple anchoring points also distribute the mechanical forces exerted on the septum, reducing the risk of tissue damage and enhancing the longevity and efficacy of the device. By employing this innovative design, the leadless ventricular pacemaker offers a significant advancement in cardiac rhythm management, providing a minimally invasive, durable, and efficient solution for patients requiring ventricular pacing. In some examples, the folded design is able to extend or lengthen after it rotates towards the apex of the right ventricle. In this example, there can be electrodes positioned near the high septum of the right ventricle with other electrodes positioned at the apex of the right ventricle. The electrodes can be spaced 60 mm apart. In other examples, the electrodes can be spaced up to 80 mm or even 120 mm apart. This is particularly useful when delivering defibrillation between these electrodes. In some examples, several elongated subunits are positioned parallel to each other and designed to lay along the interventricular septum. The proximal portion of the elongated leadless pacemaker device designed to facilitate anchoring to the high septum of the right ventricle. Next, the subunits rotate towards the apex and lay across the interventricular septum towards the apex. The device can then extend to position electrodes into the apex of the right ventricle.

In the field of implantable medical devices, inter-device communication maybe important for ensuring synchronized function and optimizing patient outcomes. Optionally, any of the leadless ventricular pacemakers herein, implanted within the septum of the right ventricle, may be designed to communicate with a separate master device that controls various other devices, such as a subcutaneous defibrillator or a cardiac resynchronization therapy (CRT) device. The communication between the leadless pacemaker and the master device can be achieved through various mechanisms, each with its own advantages and specific use cases.

One common method of communication is through wireless radiofrequency (RF) signals. RF communication is reliable and allows for high-speed data transmission over relatively short distances within the body. This method is often used in modern medical devices due to its robustness and ability to handle complex data streams. Another mechanism is inductive coupling, where communication is achieved through magnetic fields generated by coils within each device. Inductive coupling works over short distances and does not require direct line-of-sight, making it suitable for devices implanted in different locations within the body. This method is often used for both charging and data transmission. Ultrasound communication is another innovative technique where ultrasonic waves are used to transmit data between devices. This method leverages the body's natural conductivity for sound waves and can be useful in situations where RF communication is less effective due to tissue absorption or interference. Additionally, optical communication can be utilized, where infrared or visible light is used to transfer data. This requires a clear optical path between the devices but can provide high data rates and security due to the directional nature of light. A specialized method for communication in the context of the leadless ventricular pacemaker is the use of sub-threshold electrical pulses. The pacemaker can deliver sub-threshold pulses, which are not strong enough to cause a cardiac contraction but can be detected by the master device. This method leverages the existing electrical pathways and the sensitivity of the receiving device to these small signals, providing a low-energy and effective means of communication.

Finally, bio-impedance modulation is a technique where the impedance of the tissue is altered in a controlled manner to encode data. The receiving master device can detect these changes in impedance and decode the transmitted information. This method takes advantage of the natural properties of biological tissues and can be highly energy-efficient.

Optionally, any of the devices described and shown herein may include one or more of the following features.

For example, in some examples, an entire device has tines that help anchor the device into the interventricular septum. These tines may also be used to pace the ventricle.

In some examples, the device folds before the distal anchor is deployed. In other examples, the device may fold after the distal anchor is employed. For example, after anchoring, pulling the sheath back will enable the device to fold. The device may fold on itself a single time—so that there are two elongated members roughly side by side.

In some aspects, there is a hinge just distal to the superior anchoring mechanism that enables the device to rotate and lay on the interventricular septum.

In some examples, the leadless pacemaker is designed to fold before the distal anchor is deployed, while in other examples, the device may fold after the distal anchor is employed. This folding mechanism enhances the stability and positioning of the device within the interventricular septum. The folding process may be facilitated by retracting the delivery sheath, which enables the device to fold. The pacemaker may fold on itself a single time, resulting in two elongated members positioned side by side, or it may fold multiple times to further compact its structure.

In one example, the leadless pacemaker folds before the distal anchor is deployed. The device includes a flexible hinge or joint mechanism that allows it to bend at predetermined points. This pre-deployment folding can be controlled via the delivery sheath. As the sheath is retracted, the device folds on itself, positioning the elongated members side by side. This configuration ensures that the device occupies minimal space during the initial positioning and deployment phase.

In another example, the device may fold after the distal anchor is deployed. Once the distal anchor is securely embedded into the septal tissue, retracting the sheath or applying a pulling force causes the device to fold. The folding mechanism may involve a series of hinges or flexible joints that enable the device to bend at specific locations. This post-deployment folding ensures that the device is stably anchored before compacting its structure.

The leadless pacemaker may be designed to fold multiple times to enable more of the device to be implanted along the interventricular septum. The device may have a series of hinges or segmented sections that allow it to fold in a concertina or accordion-like manner. This multiple folding mechanism ensures that the device can be compacted into a smaller space, enhancing its stability and reducing the risk of displacement.

In some examples, the diameter of the leadless pacemaker devices herein may be between about three and eight millimeters (3-8 mm), making it suitable for implantation within the heart's conduction system. The device's elongated structure is segmented to allow for controlled folding. Each segment may include one or more pacing electrodes, anchoring mechanisms, or communication modules to ensure optimal performance.

The folding process is controlled by retracting the delivery sheath. As the sheath is pulled back, the device is allowed to fold at its pre-determined hinge points. This controlled folding ensures that the device aligns properly within the septal tissue and remains securely anchored.

In one example, the device folds on itself a single time, resulting in two elongated members positioned roughly side by side. This configuration provides a compact profile while maintaining the device's stability and functionality.

In another example, the device folds multiple times to further compact its structure. The multiple fold configuration may involve three or more folds, positioning the elongated segments in a stacked or side-by-side arrangement. This configuration enhances the device's stability and minimizes its spatial footprint within the heart.

The device includes both proximal and distal anchoring mechanisms to ensure secure placement. The distal anchor, which may be a screw, tines, or barbs, is deployed first to secure the device. The proximal anchor, which may also be a screw or tines, is then deployed to complete the anchoring process. The folding mechanism ensures that both anchors remain securely embedded in the septal tissue.

The leadless pacemaker is constructed from biocompatible materials that ensure its durability and flexibility. The hinges and joints are designed to withstand the mechanical stresses of folding and unfolding while maintaining their structural integrity. This design ensures that the device remains functional and stable throughout its lifespan.

The leadless pacemaker includes various mechanisms to enable it to fold and align correctly within the interventricular septum. These mechanisms ensure the device occupies minimal space and maintains stable positioning. The folding can be passive, using pre-stressed materials, or active, utilizing external forces. Here are several potential mechanisms:

In one example, the device includes a spring mechanism pre-loaded to fold the device once it is freed from the delivery sheath. As the sheath is retracted, the stored energy in the spring causes the device to fold at predetermined points.

In one example, the device is constructed with nitinol, a shape-memory alloy, used to create hinges or segments. Nitinol can be pre-set to bend at specific points when exposed to body temperature or an electrical stimulus.

In one example, the device includes an active force mechanism involving the use of external forces, such as hydraulic or pneumatic systems, to fold the pacemaker. A hydraulic or pneumatic actuator within the device exerts force on the segments, causing them to fold.

In one example, the device incorporates magnetic components that interact with an external magnetic field to induce folding. Once the device is deployed, an external magnet creates a magnetic field that causes the internal magnets to align and fold the device.

In one example, the device includes a controlled release mechanism that gradually frees pre-tensioned segments or springs. As the delivery sheath is retracted, the controlled release mechanism sequentially releases the tensioned segments, causing the device to fold.

In one example, the device utilizes electroactive polymers (EAPs) to create segments that bend or contract when an electric current is applied. Upon deployment, an electrical signal is sent to the EAPs, causing them to change shape and fold the device.

In one example, the device is designed to fold in a sequential manner, with each segment folding in a predetermined order. As the sheath is retracted, each segment is triggered to fold in sequence, ensuring that the device compacts efficiently.

In one example, the device incorporates elastic bands or strips integrated into the structure, pre-set to fold the device upon deployment. When the sheath is retracted, the elastic bands contract, folding the device into its compact configuration.

In one example, the device includes a miniaturized gear mechanism built into the structure, designed to fold the segments when activated. Upon deployment, the gears are engaged and rotated, causing the segments to fold into place.

In one example, an elongated delivery wire is used to screw the distal end of the leadless pacemaker into the interventricular septum. This ensures secure anchoring of the device. Once the distal end is anchored, the delivery wire must be released from the pacemaker. Here are several potential mechanisms for achieving this:

In one example, the delivery wire is severed using electric energy. The wire is designed with a specific section that can be heated through an electric current, causing it to melt or break, thereby detaching the delivery wire from the pacemaker.

In one example, a mechanical screw release mechanism is employed. The delivery wire is connected to the pacemaker via a threaded section. Once the distal end is anchored, the delivery wire is rotated in the opposite direction, unscrewing and releasing it from the device.

In one example, the delivery wire is released using a hydraulic or pneumatic mechanism. A small chamber within the pacemaker is pressurized with fluid or air, causing a pin or other securing element to retract, thereby releasing the delivery wire.

In one example, the release mechanism is magnetic. The delivery wire is held in place by a magnetic lock within the pacemaker. When an external magnetic field is applied, the magnetic lock is disengaged, allowing the delivery wire to be released.

In one example, a chemical release mechanism is used. The delivery wire is coated with a substance that dissolves or reacts when exposed to a specific chemical agent delivered through the catheter, causing the wire to break and release.

In one example, the delivery wire includes a biodegradable section that naturally degrades over time after implantation, releasing the wire from the pacemaker.

In one example, the delivery wire is secured by a friction lock. Once the distal end is anchored, the friction lock is released by retracting a sheath or applying a force that reduces friction, allowing the wire to be detached.

In one example, the delivery wire uses a snap-fit connection. The wire is snapped into place within the pacemaker and can be released by applying a specific force or manipulating the wire to disengage the snap-fit connection.

In one example, a shear pin is used to hold the delivery wire in place. Once the distal end is anchored, a force is applied to shear the pin, releasing the wire from the device.

In one example, the delivery wire is connected to the pacemaker via a component that expands when heated. Applying heat causes thermal expansion, loosening the connection and releasing the wire.

Verification of Placement: Accurate placement and secure anchoring of the pacemaker are verified using fluoroscopy and ICE. Fluoroscopic imaging offers a real-time view of the device's position, while ICE provides detailed images of the septal wall, confirming the proper embedding of the screw or tines.

In one example, the leadless ventricular pacemaker includes a labeling system designed to assist the proceduralist in accurately determining the orientation of the device during deployment. This labeling system can be visualized through fluoroscopy or intracardiac echocardiography (ICE), ensuring that the device is correctly positioned and rotated into the interventricular septum.

The labeling system may include one or more radiopaque markers or labels strategically placed on the pacemaker. These markers are designed to form a specific shape or pattern when viewed under fluoroscopy or ICE, providing clear visual cues to the proceduralist. A commonly used shape for such markers is a “C” shape, similar to those employed in transcatheter aortic valve replacement (TAVR) procedures.

### Fluoroscopy and ICE Visualization

Optionally, the devices herein may include one or more radiopaque markers that are visible under fluoroscopy. These markers form a “C” shape when the device is oriented correctly. The markers are positioned on the device such that they remain visible during the entire deployment process, from insertion to final positioning. In addition to radiopaque markers, the device may include echogenic markers that are visible under Intracardiac Echocardiography (ICE). These markers provide additional visual confirmation of the device's orientation. The echogenic markers are designed to produce distinct echoes that form recognizable patterns, such as a “C” shape, ensuring clear visualization.

Initial Positioning: The proceduralist advances the leadless pacemaker into the right ventricle using the delivery system, guided by fluoroscopy or ICE. The radiopaque or echogenic markers become visible on the imaging screen, providing real-time feedback on the device's orientation. Orientation Determination: As the device approaches the target location in the high mid-septum, the proceduralist looks for the “C” shape formed by the markers. The presence of the “C” shape indicates that the device is oriented correctly for rotation and folding into the interventricular septum. Rotation and Folding: Using the delivery system, the proceduralist rotates the device until the “C” shape is clearly visible and properly aligned with the septum. Once aligned, the proceduralist initiates the folding mechanism, allowing the device to fold into the septal wall. Confirmation: The proceduralist confirms the final positioning and anchoring of the device by verifying the marker pattern on the imaging screen. Additional fluoroscopic or ICE imaging may be used to ensure that the device is securely anchored and properly aligned with the conduction system.

In one example, the elongated leadless ventricular pacemaker includes a hinge mechanism positioned between the anchoring portion and the elongated portion of the device. This hinge mechanism enables controlled rotation and alignment of the device within the interventricular septum, ensuring optimal positioning for effective pacing. The hinge mechanism is constructed from durable, biocompatible materials such as nitinol, known for its shape-memory properties and flexibility. The hinge allows the elongated portion of the pacemaker to rotate relative to the anchoring portion, facilitating precise positioning within the heart. Upon deployment, the hinge mechanism enables the device to bend at specific points, allowing the elongated portion to align correctly with the interventricular septum. This controlled rotation ensures that the device can fold into its intended configuration, enhancing stability and reducing the risk of displacement. The hinge may be constructed from nitinol, which bends at specific points when exposed to body temperature or an electrical stimulus, facilitating the folding process. The hinge mechanism may include a spring-loaded component that activates upon release from the delivery sheath, causing the device to rotate and align correctly. In another example, the hinge mechanism is actuated by hydraulic or pneumatic pressure, providing controlled movement and alignment of the device within the septum. The hinge mechanism may also include magnetic components that interact with an external magnetic field, allowing for precise control over the rotation and alignment of the device.

The hinge mechanism ensures that the elongated portion of the pacemaker can be accurately positioned within the interventricular septum, providing effective pacing and reducing the risk of device migration. By enabling controlled rotation and alignment, the hinge mechanism enhances the stability of the device and ensures that the pacing electrodes are optimally placed for capturing the heart's conduction system. The hinge mechanism is particularly useful in complex cardiac procedures where precise positioning of the pacemaker is critical for effective therapy. This feature allows the device to adapt to the unique anatomical structures of each patient, ensuring personalized and effective cardiac resynchronization therapy.

By incorporating a hinge mechanism between the anchoring portion and the elongated portion, the leadless ventricular pacemaker can achieve precise positioning and alignment within the interventricular septum. This ensures effective pacing and enhances the overall stability and functionality of the device.

To allow the device to deliver defibrillation shocks, several key features may be added in various examples. In one example, a high-capacity battery capable of storing and delivering the energy required for defibrillation shocks may be integrated in the device. This battery may provide the necessary electrical energy for high-voltage shock delivery. Additionally, one or more capacitors may be included to store and discharge the high-voltage energy needed for defibrillation, allowing for the rapid release of stored energy to achieve the required defibrillation waveform.

The device may also include shock electrodes configured for delivering high-voltage shocks, strategically positioned within the heart. In one example, this includes an extendable electrode to the RV apex and an extendable tail electrode to the coronary sinus, ensuring effective energy delivery across the heart for defibrillation. These electrodes may provide a large shock vector from the RV base to the RV apex and an alternative shock vector extending into the coronary sinus.

To optimize the defibrillation process, various examples herein may incorporate circuitry capable of generating biphasic and triphasic shock waveforms, providing varying waveforms that can improve shock success rates. Advanced control electronics may be useful in these examples for managing the timing, energy level, and waveform of the shocks, ensuring a precise and effective defibrillation process. Sensing and detection algorithms may be incorporated in different examples to identify life-threatening arrhythmias and automatically trigger the delivery of defibrillation shocks.

Additionally, in any of the examples herein, wireless communication capabilities may be added to allow for remote monitoring and control, enabling healthcare providers to monitor and adjust device settings as needed. Another useful feature that may be included in any of the examples is the capability to deliver anti-tachycardia pacing (ATP) before escalating to defibrillation shocks. This feature provides a less aggressive initial treatment for certain arrhythmias, potentially avoiding the need for a shock.

Integrating these features involves several steps, which may vary across different examples. In one example, the high-energy battery and capacitors may be incorporated into the main body of the device or within the RA space if size permits. Shock electrodes may be designed to be deployable and extendable to the RV apex and into the coronary sinus. In another example, shock waveform generator circuitry may be provided and integrated within the control unit of the device. Enhanced control electronics with algorithms for arrhythmia detection and shock timing may be embedded within the device's microprocessor in yet another example. Various examples may add telemetry capabilities for remote monitoring and adjustment of device settings. Finally, ATP algorithms may be implemented in some examples to ensure the device can switch seamlessly between pacing and shock delivery.

In one example, the device includes a sophisticated mechanism for delivering defibrillation shocks by extending the distal electrode after initial anchoring. The process begins with securely anchoring the device in the septum, ensuring a stable and reliable position within the heart. This anchoring can be achieved using screws or tines that firmly secure the device in place, providing a stable foundation for subsequent actions.

Once the device is anchored, it undergoes a controlled flip and rotation to align the distal portion properly within the heart. This movement is useful for positioning the device optimally, ensuring that the subsequent extension of the electrode is both precise and effective. Following this alignment, the device extends or stretches an electrode towards the RV apex. This extension is designed to create a sufficient shock vector, enabling the delivery of high-voltage shocks from the RV base to the RV apex. The extended electrode ensures that the shock covers a significant portion of the heart, thereby enhancing the effectiveness of defibrillation.

The mechanism for extending the electrode can involve a telescoping design, where the electrode elongates as it is pushed towards the RV apex. Alternatively, the electrode could unfurl or be driven into position using a controlled deployment system. Throughout this process, the device must maintain its stability to prevent dislodgement. This stability is achieved through the initial anchoring system, which holds the device securely in place while the distal electrode is extended.

In addition to extending the electrode towards the RV apex, some examples incorporate an advanced feature that includes an extendable “tail” which can be navigated and advanced into the coronary sinus. This allows for the creation of an alternative shock vector, enabling the delivery of defibrillation shocks from the RV septum to an electrode strategically positioned within the coronary sinus. By extending the tail electrode into the coronary sinus, the device can provide a more comprehensive and adaptable defibrillation strategy, ensuring effective shock delivery across different regions of the heart.

This feature enhances the device's versatility, allowing it to cater to various anatomical and clinical requirements. The extendable tail mechanism ensures precise placement of the electrode within the coronary sinus, providing a stable and effective shock pathway. The ability to deliver shocks from the RV septum to the coronary sinus electrode enhances the overall efficacy of the defibrillation process, offering an additional treatment vector that can be critical for restoring normal heart rhythm.

In some examples, the device also integrates high-capacity batteries and defibrillator coils within the RV septum or coronary sinus, or both. These components are useful for storing and releasing the high-voltage energy required for defibrillation. By embedding the batteries and coils in these strategic locations, the device minimizes energy loss and enhances efficiency, ensuring that the shocks are delivered effectively. The batteries may be configured to fold or reconfigure into a new shape once in the patient's heart or before entering the patient's heart to minimize space requirements. Batteries and their containers may be flexible to enable reconfiguring or rigid with hinge or other translational and rotational mechanisms.

In addition to the RV apex and coronary sinus, the device can incorporate other potential locations and methods for delivering shocks: The device can be equipped with circuitry capable of generating biphasic and triphasic shock waveforms. Biphasic shocks, which involve delivering two phases of current in opposite directions, and triphasic shocks, with three phases, can optimize the defibrillation process by reducing the energy required and increasing the likelihood of successful defibrillation. A multi-electrode array can be integrated into the device, allowing for simultaneous or sequential shock delivery from multiple locations within the heart. For example, electrodes can be positioned along the interventricular septum, within the RV, and within the left ventricle (LV). This approach provides multiple shock vectors, enhancing the overall defibrillation success. An additional electrode can be extended into the left ventricle (LV). This can be particularly useful for creating a shock vector that spans from the RV septum to the LV, covering more myocardial tissue and increasing defibrillation efficacy. A subcutaneous electrode can be included as part of the system, placed just under the skin. This electrode can work in conjunction with the internal electrodes to provide an external shock vector, enhancing the defibrillation capability. For certain patients, an epicardial electrode placed on the outer surface of the heart can be utilized. This provides another vector for shock delivery, potentially increasing the success rate of defibrillation. An implantable coil can be integrated into the RV septum or another strategic location. This coil can store and release high-energy shocks, providing a robust method for delivering defibrillation.

The devices herein may incorporate various advanced methods for delivering defibrillation shocks, each method tailored to enhance effectiveness and adaptability to different clinical needs. These methods may be implemented in different examples as follows, where any of these examples may be combined with each other or other examples:
Multi-Point Shocking Example: In this example, the device utilizes a multi-electrode array positioned at different locations within the heart. Electrodes are strategically placed along the interventricular septum, in the right ventricle (RV), left ventricle (LV), and the coronary sinus. This configuration allows for simultaneous or sequential shock delivery from multiple locations, creating extensive shock vectors that cover more myocardial tissue, thus enhancing the overall success of defibrillation.
Subcutaneous Shocking Example: This example includes a subcutaneous electrode placed just under the skin. This electrode works in conjunction with internal electrodes to provide an external shock vector. By incorporating subcutaneous shocking, the device enhances its defibrillation capability by adding an additional pathway for shock delivery, which can be particularly useful in certain patient populations.
Epicardial Shocking Example: In this example, an electrode is placed on the outer surface of the heart (epicardium). This electrode can be attached during a surgical procedure, providing another vector for shock delivery. Epicardial shocking offers an additional approach that can increase the success rate of defibrillation by covering areas not easily reached by internal electrodes.
Biphasic Shocking Example: This example features advanced circuitry capable of generating biphasic shock waveforms. Biphasic shocks deliver two phases of current in opposite directions, optimizing the defibrillation process by reducing the energy required and improving shock success rates. This method is particularly effective in minimizing potential damage to the myocardial tissue while ensuring effective defibrillation.
Triphasic Shocking Example: Building on the biphasic method, this example uses circuitry that generates triphasic shock waveforms, which deliver three phases of current. Triphasic shocking further reduces energy requirements and enhances defibrillation efficacy compared to biphasic shocks, providing an even more refined approach to shock delivery.
Helical Coil Shocking Example: In this example, the device integrates a helical coil electrode within the RV septum or another strategic location within the heart. The helical coil provides concentrated and effective shock delivery, ensuring that the energy is directed precisely where needed to achieve successful defibrillation.
Far-Field Shocking Example: This example utilizes electrodes placed at a distance from each other to create a broad shock vector. Electrodes can be positioned in the RV apex and coronary sinus, or other distant locations within the heart. Far-field shocking ensures that a larger area of the heart is covered by the shock, improving overall defibrillation outcomes.
Intrathoracic Shocking Example: In this example, electrodes are placed within the thoracic cavity, such as on the diaphragm or other intrathoracic structures. This method provides a comprehensive shock vector that covers a significant portion of the heart and surrounding structures, enhancing the effectiveness of defibrillation.
Sequential Shocking Example: This example involves delivering shocks in a sequential manner using multiple electrodes. The control electronics within the device are programmed to time the shocks in a specific sequence, allowing for targeted shock delivery that can be optimized for different types of arrhythmias. This method provides flexibility in managing various cardiac conditions.
Burst Shocking Example: In this example, the device delivers a series of rapid, low-energy shocks over a short period, known as burst shocking. The circuitry generates a burst of shocks that can effectively terminate certain types of arrhythmias without the need for a single high-energy shock. This method is particularly useful for treating less severe arrhythmias while minimizing discomfort and potential damage to the myocardial tissue.

By incorporating these features, the device is capable of both flipping and rotating after anchoring to extend the distal electrode towards the RV apex. This ensures effective shock delivery from the RV base to the RV apex, covering a broad area of the heart. The stable anchoring during the extension process ensures that the device remains securely positioned, allowing for precise and effective defibrillation. This comprehensive approach enhances the device's capability to provide therapy for patients with heart failure and arrhythmias, making it a versatile and effective solution in cardiac care. The device may be capable of both pacing and delivering defibrillation shocks, providing comprehensive therapy for patients with heart failure and arrhythmias.

The device can be designed to deliver anti-tachycardia pacing (ATP) from both the coronary sinus (CS) and the right ventricle (RV), with advanced capabilities to analyze paced morphology and post-pacing intervals. This analysis can guide subsequent ATP deliveries and inform the timing, morphology, and type of shocks delivered. Here are the detailed descriptions of these capabilities in different examples:

In some examples, the device may deliver ATP from both the coronary sinus (CS) and the right ventricle (RV). The device is equipped with electrodes positioned in the CS and RV. These electrodes can deliver precise pacing stimuli to terminate tachyarrhythmias. Delivering ATP from two distinct sites increases the likelihood of effectively terminating arrhythmias by capturing different parts of the heart's conduction system. The device continuously monitors the morphology of the paced electrocardiogram (ECG) during ATP delivery. Sensors integrated with the electrodes analyze the morphology of the ECG signal during and after pacing. The morphology of the paced ECG provides valuable information about the effectiveness of ATP, helping to adjust subsequent pacing parameters for improved arrhythmia termination.

In this and other examples, after delivering ATP, the device measures the interval between pacing stimuli and the return of intrinsic heartbeats. The device's control electronics record the time between the last pacing pulse and the next spontaneous heartbeat. This post-pacing interval data helps determine the success of ATP and guides adjustments for future ATP deliveries. Using the data from post-pacing interval measurements, the device adjusts the timing and parameters of subsequent ATP deliveries. Algorithms within the device analyze the post-pacing interval data and modify ATP parameters, such as pacing rate and pulse duration. Optimizing ATP delivery based on real-time data enhances the effectiveness of arrhythmia termination.

In one example, adaptive shock is delivered based on ATP data. The device uses data from paced morphology and post-pacing intervals to adjust the timing and morphology of delivered shocks. The control electronics analyze the ATP data and determine the optimal time to deliver a shock, as well as whether a biphasic or triphasic waveform is most appropriate. By tailoring the shock parameters based on ATP data, the device can deliver more effective defibrillation therapy with reduced energy requirements. The device decides whether to use biphasic or triphasic shocks or other shock based on the effectiveness of ATP and the morphology of the paced ECG. Advanced algorithms evaluate the ATP data and select the appropriate shock type for each situation. Choosing the right shock type enhances defibrillation success rates and reduces potential myocardial damage.

Another example may combine ATP delivery and defibrillation shocks. This example integrates ATP delivery with defibrillation shocks, using ATP data to guide both therapies. The device delivers ATP as the first line of therapy and continuously monitors the results. If ATP is unsuccessful, it uses the gathered data to inform subsequent shock delivery. Combining ATP and shock therapy in a data-driven manner ensures a comprehensive approach to arrhythmia management, maximizing treatment effectiveness while minimizing unnecessary shocks. The device continuously adjusts ATP and shock parameters in real-time based on ongoing data collection. The control electronics and embedded software analyze data on the fly, making instant adjustments to therapy delivery. Real-time feedback and adjustment provide dynamic and responsive treatment, improving patient outcomes.

Electrodes are strategically placed in the CS and RV to deliver precise ATP and gather detailed ECG data. These electrodes are capable of both pacing and sensing, providing comprehensive data for analysis. Continuous ECG monitoring allows the device to analyze the morphology of paced beats, using this information to assess ATP effectiveness. The time interval between pacing and the return of intrinsic heartbeats is recorded and analyzed, guiding subsequent therapy. Algorithms within the device use data from ATP deliveries to adjust pacing parameters, shock timing, and shock morphology, ensuring optimized treatment. The control electronics are designed for high-speed data processing, enabling real-time analysis and adjustments. Advanced software algorithms are embedded within the device to interpret data and make therapeutic decisions dynamically.

By integrating these advanced features, the device becomes capable of delivering tailored and highly effective anti-tachycardia pacing and defibrillation shocks. This comprehensive approach leverages real-time data to optimize therapy, providing robust and adaptive cardiac care for patients with arrhythmias.

It will be appreciated that elements or components shown with any example herein are exemplary for the specific example and may be used on or in combination with other examples disclosed herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A leadless ventricular pacemaker device, comprising:
an elongated member configured to be advanced through a tubular member into a patient's heart;
a conformational change mechanism configured to reconfigure the elongated member from an elongated shape to a folded state upon exiting the tubular member; and
a plurality of electrodes on the elongated member and configured to deliver unipolar or bipolar pacing to tissue within the patient's heart,
wherein the device is configured to be implanted into a septum of a right ventricle within or adjacent to a native conduction system of the patient's heart.

2. The device of claim 1, wherein the conformational change mechanism is configured to transition the elongated member from a compact to a reconfigured state upon exiting the tubular member.

3. The device of claim 1, further comprising:
a second elongated member configured to be advanced through a tubular member and deployed near or within a right atrium of the patient's heart; and
a second conformational change mechanism configured to reconfigure the second elongated member upon the release from the tubular member to facilitate a conformational change of the second elongated member, minimizing a profile of the second elongated member during delivery and ensuring robust anchoring and electrode contact once deployed.

4. The device of claim 1, wherein the plurality of electrodes comprises at least three electrodes configured to penetrate and embed into tissue and deliver electrical pulses in a variety of pacing configurations.

5. The device of claim 1, further comprising an anchoring system configured to provide thermal anchoring of the elongated member to tissue, including one or more of resistive heating, radiofrequency (RF) ablation, laser ablation, and ultrasound ablation to securely anchor the device to the tissue.

6. A leadless ventricular pacemaker, comprising:
an elongated member configured to be implanted within a septum of a right ventricle of a patient's heart; and
a plurality of anchors on the elongated member and spaced apart from one another such that the anchoring mechanisms are positioned at at least two locations along the septum and spaced apart between about ten and forty millimeters (10-40 mm).

7. The pacemaker of claim 6, wherein the anchors comprise a proximal anchor configured to be positioned in a high interventricular septum location and a distal anchor configured to be positioned lower in the septum such that the proximal and distal anchors are spaced between about ten and forty millimeters (10-40 mm).

8. The pacemaker of claim 6, further comprising a hinge mechanism on each of opposite ends of the elongated member.

9. The pacemaker of claim 8, wherein each hinge mechanism comprises nitinol utilizing shape-memory properties for bending and rotation upon deployment.

10. A leadless ventricular pacemaker device, comprising:
an elongated member configured to be advanced through a tubular member into a patient's heart;
a conformational change mechanism configured to transition the elongated member from an elongated to a reconfigured state upon exiting the tubular member, wherein at least two elongated subunits automatically position themselves relatively parallel to each other; and
a plurality of electrodes on the elongated member, configured to deliver pacing to tissue within the patient's heart;
wherein the device is configured to be implanted into the septum of the right ventricle, within or adjacent to the native conduction system of the patient's heart.

11. The device of claim 10, wherein the device is configured to deliver a defibrillation shock to cardiovert a dangerous rhythm within the patient's heart.

12. The device of claim 10, wherein the device communicates with a separate implantable device located elsewhere in the body through sub-threshold pacing pulses.

13. The device of claim 10, wherein the plurality of electrodes comprise at least three electrodes configured to penetrate and embed into tissue and deliver electrical pulses in a variety of pacing configurations.

14. The device of claim 10, further comprising an anchoring system configured to provide thermal anchoring of the elongated member to tissue, including one or more of resistive heating, radiofrequency (RF) ablation, laser ablation, and ultrasound ablation to securely anchor the device to the tissue.

15. The device of claim 1, further comprising a power source coupled to the electrodes to deliver electrical energy to the electrodes to deliver the pacing.

16. The device of claim 15, further comprising a controller coupled to the power source to control one or more parameters of the pacing delivered by the electrodes.

17-21. (canceled)

22. A method of implanting a leadless ventricular pacemaker device, comprising:
advancing an elongated member through a tubular member into a patient's heart;
reconfiguring the elongated member from an elongated shape to a folded state after exiting the tubular member using a conformational change mechanism, the elongated member comprising a plurality of electrodes configured to deliver unipolar or bipolar pacing to tissue within the patient's heart; and
implanting the elongate member into a septum of a right ventricle within or adjacent to a native conduction system of the patient's heart.

23. The method of claim 21, further comprising facilitating the transition of the elongated member from a compact to a reconfigured state upon exiting the delivery sheath using the conformational change mechanism.

24. The method of claim 21, further comprising:
advancing a second elongated member through a tubular member into a right atrium of the patient's heart; and
deploying the second elongated member within the right atrium, whereupon the second elongated member undergoes a conformational change upon the release from the tubular member, minimizing a profile of the second elongated member during delivery and ensuring robust anchoring and electrode contact once deployed.

25. The method of claim 21, further comprising penetrating and embedding at least three electrodes on the elongated member into septal tissue within the patient's heart configured for delivering electrical pulses in a variety of pacing configurations.

25-30. (canceled)
